(19) United States
(12) Patent Application Publication
(10) Pub. No.: US 2015/0157825 A1
(43) Pub. Date: Jun. 11, 2015

(54) LOCKING ASSEMBLY FOR CONNECTING TWO RESPIRATORY TUBES

(71) Applicant: HSINER Co., Ltd., Taichung City (TW)

(72) Inventor: Eric CHANG, Taichung City (TW)

(73) Assignee: HSINER Co., Ltd., Taichung City (TW)

(21) Appl. No.: 14/097,864

(22) Filed: Dec. 5, 2013

(51) Int. Cl. A61M 16/08

(52) U.S. Cl. CPC A61M 16/0816; A61M 16/0875

(57) ABSTRACT

A locking assembly is adapted for connecting a first respiratory tube and a second respiratory tube. The first respiratory tube has an outer surrounding surface. The locking assembly includes a sleeve and a coupling unit. The sleeve is adapted to be sleeved fittingly on the first respiratory tube and has an inner surface, an outer surface, and a first locking portion. The coupling unit includes a connecting tube and a locking component that has a second locking portion. The second locking portion is pivotable relative to the connecting tube between a locked position and an unlocked position.

### BACKGROUND OF THE INVENTION

1. Field of the Invention

This invention relates to a locking assembly for a breathing assisting equipment, more particularly to a locking assembly for connecting two respiratory tubes of the breathing assisting equipment.

2. Description of the Related Art

Taiwanese Utility Patent No. M424954 discloses a breathing mask that includes a mask body, and a first L-shaped respiratory tube. The first L-shaped respiratory tube is adapted to be connected to a second respiratory tube that is connected to an air supply to deliver a gas, e.g., oxygen, from the second respiratory tube through the first respiratory tube to the mask body.

According to provisions of ISO5356-1, the first respiratory tube and the second respiratory tube must meet the 22/15 mm specification. In general, the first respiratory tube is directly connected to the second respiratory tube by interference fit. The interference fit connection may be convenient for some users or operators because the first and second respiratory tubes can be easily separated by directly pulling one of the respiratory tubes away from the other one. However, the tight connection will gradually become weak and the respiratory tubes may easily disengage from each other after the two respiratory tubes are frequently connected with and disconnected from each other. The reliability of the connection is thus affected.

Therefore, there is a need in the art to provide locking assembly for reliably connecting two respiratory tubes used in the breathing mask.

### SUMMARY OF THE INVENTION

Therefore, the object of the present invention is to provide a locking assembly for connecting two respiratory tubes that can overcome the aforesaid drawback of the prior art.

According to this invention, there is provided a locking assembly adapted for connecting a first respiratory tube and a second respiratory tube The first respiratory tube has an outer surrounding surface. The locking assembly comprises a sleeve and a coupling unit. The sleeve is adapted to be sleeved fittingly on the first respiratory tube, and has an inner surface to contact the outer surrounding surface of the first respiratory tube, an outer surface opposite to the inner surface, and a first locking portion that is formed on the outer surface. The coupling unit includes a connecting tube and a locking component The connecting tube is adapted to be sleeved fittingly in the second respiratory tube. The sleeve is inserted fittingly into the connecting tube. The locking component has a connecting part connected to the connecting tube, and a second locking portion connected to the connecting part and pivotable relative to the connecting tube at the connecting part between a locked position, where the second locking portion engages the first locking portion, and an unlocked position, where the second locking portion is disengaged from the first locking portion.

### BRIEF DESCRIPTION OF THE DRAWINGS

Other features and advantages of the present invention will become apparent in the following detailed description of the preferred embodiment of this invention, with reference to the accompanying drawings, in which:

FIG. 1 is a fragmentary exploded partly sectional view showing the preferred embodiment of a locking assembly of the present invention, that is connected to first and second respiratory tubes;

FIG. 2 is a fragmentary exploded partly sectional view illustrating a sleeve and a coupling unit of the preferred embodiment respectively connected to the first and second respiratory tubes by interference fit; and

FIG. 3 is a fragmentary partly sectional view illustrating the sleeve and the coupling unit of the preferred embodiment after being assembled together.

### DETAILED DESCRIPTION OF THE PREFERRED EMBODIMENT

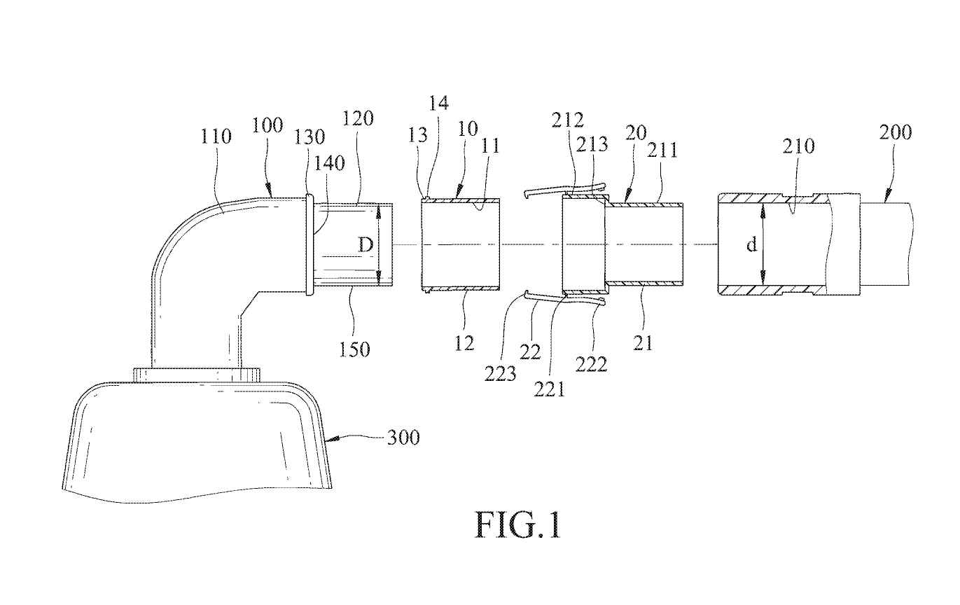

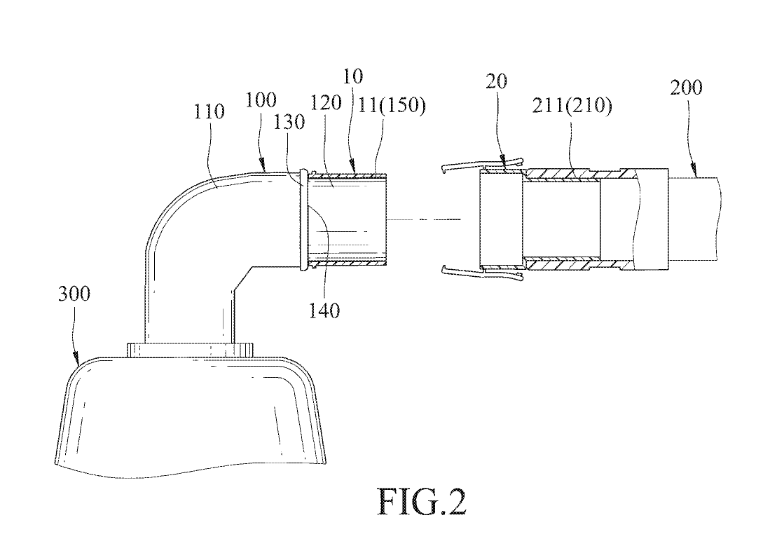

Referring to FIGS. 1 and 2, a locking assembly of the preferred embodiment according to the present invention is used to interconnect a first respiratory tube 100 and a second respiratory tube 200. The first respiratory tube 100 is connected to a breathing mask 300 and has a bent tube segment 110, a straight tube segment 120 connected to the bent tube segment 110, a flange 130 extending radially and outwardly from a junction of the bent tube segment 110 and the straight tube segment 120, and an annular shoulder surface 140 located between the junction of the flange 130 and the straight tube segment 120 and facing the straight tube segment 120. The straight tube segment 120 is formed with an outer surrounding surface 150 that has an outer diameter (D). The second respiratory tube 200 has an inner surrounding surface 210 having an inner diameter (d). The inner and outer diameters meet the 22/15 mm specification of ISO5356-1.

The locking assembly of this embodiment includes a sleeve 10 and a coupling unit 20. The sleeve 10 is to be sleeved fittingly on the straight tube segment 120 of the first respiratory tube 100, and has an inner surface 11 to contact the outer surround surface 150 of the first respiratory tube 100, an outer surface 12 that is opposite to the inner surface 11, a first locking portion 13 that is formed on the outer surface 12, and an annular projection 14 that projects radially and outwardly from the outer surface 12. The coupling unit 20 includes a connecting tube 21 that is to be sleeved fittingly in the second respiratory tube 200, and a pair of locking components 22 that are spaced apart from each other and that are connected to and pivotable relative to the connecting tube 21.

In this embodiment, the first locking portion 13 of the sleeve 10 is configured as an annular space and is located at one side of the annular projection 14. More specifically, the annular projection 14 cooperates with a portion of the outer surface 12 of the sleeve 10 that is opposite to the connecting tube 21 to define the first locking portion 13 so that the first locking portion 13 is formed between the annular shoulder surface 140 and the annular projection 14 when the sleeve 10 is sleeved fittingly on the straight tube segment 120 of the first respiratory tube 100.

The connecting tube 21 is configured to have a small diameter section 211 that is to be sleeved fittingly in the inner surrounding surface 210 of the second respiratory tube 200, a larger diameter section 212 that is connected to a side of the small diameter section 211 and that has an inner diameter larger than that of the small diameter section 211, and an abutment section 213 that interconnects the small diameter section 211 and the larger diameter section 212. Each of the locking components 22 is connected to the larger diameter section 212, and has a connecting part 221 connected to the larger diameter section 212 of the connecting tube 21, an operating part 222 located at one side of the connecting part 221, and a second locking portion 223 that is located at the other side of the connecting part 221 and opposite to the operating part 222. The second locking portion 223 is connected to the connecting part 221 and is pivotable relative to the connecting tube 21 at the connecting part 221. The second locking portion 223 and the first locking portion 13 are complementary to each other in structure and can be locked together. In this embodiment, the second locking portion 223 is configured as an annular protrusion that is complementary to the annular space of the first locking portion 13.

Referring to FIG. 1, in an unassembled condition, the first respiratory tube 100, the sleeve 10, the coupling unit 20, and the second respiratory tube 200 are all separate. The second locking portion 223 is pivoted toward a central axis of the connecting tube 21, and the operating part 222 is disposed away from the central axis of the connecting tube 21 which leaves an operating space between the larger diameter section 212 and the operating part 222.

Referring to FIG. 2, in use, the sleeve 10 is sleeved fittingly on the straight tube segment 120 such that a first end of the sleeve 10 abuts against the flange 130 and the inner surface 11 of the sleeve 10 fits the outer surrounding surface 150 of the first respiratory tube 100. Furthermore, the coupling unit 20 is sleeved fittingly in the inner surrounding surface 210 of the second respiratory tube 200.

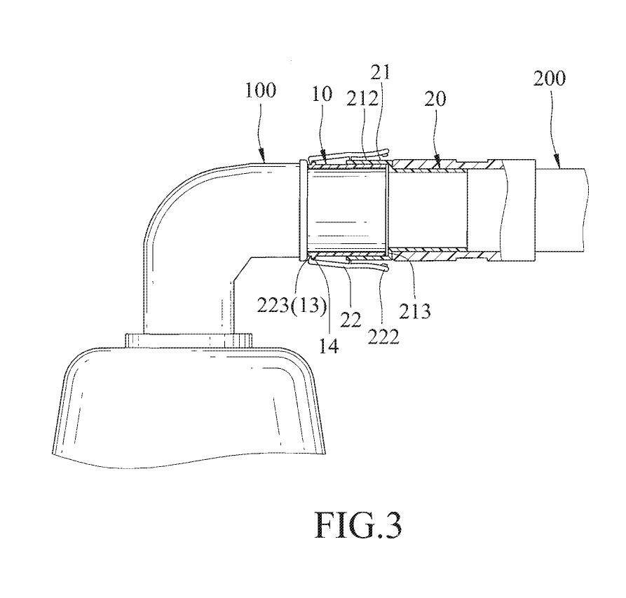

Referring to FIG. 3, the sleeve 10 that is sleeved on the first respiratory tube 100 is further inserted into the larger diameter section 212 of the connecting tube 21 such that a second end of the sleeve 10 that is opposite to the first end abuts against the abutment section 213. The locking components 22 are then resiliently pivoted such that the second locking portion 223 engages the first locking portion 13 in a locked position. In the locked position, the annular projection 14 abuts against the second locking portion 223 and prevents the second locking portion 223 from disengaging. As a result, the sleeve 10 and the coupling unit 20 can be assembled together and will not be separated easily. In the locked position, the second respiratory tube 200 still could be freely rotated with respect to the first respiratory tube 100.

Upon disassembling, users or operators only have to press the operating part 222 of each of the locking components 22 to move the operating part 222 toward the larger diameter section 212, and thus, the second locking portion 223 is disengaged from the first locking portion 13. As a result, the sleeve 10 and the coupling unit 20 can be disassembled easily (back to the state shown in FIG. 2).

To sum up, with the locking assembly of this invention, connection and disconnection between the first and second respiratory tubes 100, 200 could be easily conducted, and reliable connection between the first and second respiratory tubes 100, 200 could be ensured.

While the present invention has been described in connection with what is considered the most practical and preferred embodiment, it is understood that this invention is not limited to the disclosed embodiment but is intended to cover various arrangements included within the spirit and scope of the broadest interpretation and equivalent arrangements.

What is claimed is:

1. A locking assembly adapted for connecting a first respiratory tube and a second respiratory tube, the first respiratory tube having an outer surrounding surface, said locking assembly comprising:
a sleeve adapted to be sleeved fittingly on the first respiratory tube, and having an inner surface to contact the outer surrounding surface of the first respiratory tube, an outer surface that is opposite to said inner surface, and a first locking portion that is formed on said outer surface; and
a coupling unit including
a connecting tube that is adapted to be sleeved fittingly in the second respiratory tube, said sleeve being inserted fittingly into said connecting tube, and
a locking component that has a connecting part connected to said connecting tube, and a second locking portion connected to said connecting part and pivotable relative to said connecting tube at said connecting part between a locked position, where said second locking portion engages said first locking portion, and an unlocked position, where said second locking portion is disengaged from said first locking portion.

2. The locking assembly as claimed in claim 1, wherein:
said first locking portion is configured as an annular space; and
said second locking portion is configured as an annular protrusion.

3. The locking assembly as claimed in claim 2, wherein said sleeve further has an annular projection that projects radially and outwardly from said outer surface and that cooperates with a portion of said outer surface that is opposite to said connecting tube to define said first locking portion.

4. The locking assembly as claimed in claim 3, wherein the first respiratory tube has a bent tube segment, a straight tube segment connected to the bent tube segment and formed with the outer surrounding surface, a flange extending radially and outwardly from a junction of the bent tube segment and the straight tube segment, and an annular shoulder surface facing the straight tube segment, said first locking portion being formed between the annular shoulder surface and said annular projection of said sleeve.

5. The locking assembly as claimed in claim 1, wherein said connecting tube has a small diameter section adapted to be sleeved fittingly in the second respiratory tube, a larger diameter section having an inner diameter that is larger than that of said small diameter section, and an abutment section interconnecting said small diameter section and said larger diameter section, said sleeve being inserted fittingly into said larger diameter section and abutting against said abutment section, said connecting part of said locking component being connected to said larger diameter section of said connecting tube.

6. The locking assembly as claimed in claim 1, wherein said coupling unit includes a pair of said locking components that are spaced apart from each other.
